(19) United States
(12) Patent Application Publication
(10) Pub. No.: US 2025/0290837 A1
(43) Pub. Date: Sep. 18, 2025

(54) FATIGUE LIFE PREDICTION METHOD OF MODIFIED FIELD INTENSITY APPROACH BASED ON NOTCH TYPE DIVISION

(71) Applicant: NANJING TECH UNIVERSITY, Nanjing (CN)

(72) Inventors: Xiaowei WANG, Nanjing (CN); Jiayuan GU, Nanjing (CN); Tianyu ZHANG, Nanjing (CN); Xiancheng ZHANG, Shanghai (CN); Zitong KANG, Nanjing (CN); Guanhong CHEN, Nanjing (CN); Jianming GONG, Nanjing (CN); Shandong TU, Shanghai (CN)

(21) Appl. No.: 18/660,745

(22) Filed: May 10, 2024

(30) Foreign Application Priority Data
Mar. 14, 2024 (CN) 2024102939535

(51) Int. Cl. G01N 3/02

(52) U.S. Cl. CPC G01N 3/02

(57) ABSTRACT

The disclosure relates to a technical field of engineering metals, in particular to a fatigue life prediction method based on notch type division by a modified field intensity approach, including: S1, carrying out elastic-plastic finite element analysis on a notched component to be analyzed, obtaining a stress-strain distribution, extracting a stress distribution on the most dangerous path of the notched component, and calculating a corresponding relative stress gradient; S2, dividing notch types according to an obtained relative stress gradient; S3, defining a field diameter of a corresponding notch according to different stagnation point positions; S4, according to a divided field diameter, bringing the field diameter divided into simplified one-dimensional field intensity approach models, and calculating a corresponding equivalent stress according to the divided field diameter; and S5, combining an S-N curve of a smooth material, and predicting the fatigue life of the notched component according to a calculated equivalent stress.

### TECHNICAL FIELD

The disclosure relates to a technical field of engineering metals, and in particular to a fatigue life prediction method of a modified field intensity approach based on notch type division.

### BACKGROUND

The research on notch fatigue of metal materials is an important engineering and scientific issue, involving aviation, automobile, architecture, machinery manufacturing and other fields. With the progress of modern society and the innovation of cutting-edge technology, more and more sophisticated equipment and vehicles have been developed and manufactured to meet the people's yearning for a better life, which puts forward higher requirements for the mechanical properties of key components. The structures are often designed to be more complex, and there are a lot of irregular and geometrically discontinuous structures in order to meet the diverse functional requirements of components. These geometric discontinuities will lead to local stress concentration, which will inevitably be affected by notch effect, and the parts with local stress concentration are often the most prone to fatigue failure, which brings new challenges to the reliability design and integrity evaluation of structures.

In order to solve the problem of multiaxial fatigue life evaluation of components under notch effect, the conventional field intensity approach model has a huge amount of calculation for predicting notched components, and it is difficult to define the fatigue failure zone.

### SUMMARY

Based on the above purpose, the disclosure provides a fatigue life prediction method of a modified field intensity approach based on notch type division.

The disclosure relates to a fatigue life prediction method of a modified field intensity approach based on notch type division, including following steps:
S1, carrying out elastic-plastic finite element analysis on a notched component to be analyzed, obtaining a stress-strain distribution, extracting a stress distribution on the most dangerous path of the notched component, and calculating a corresponding relative stress gradient;
S2, dividing notch types according to an obtained relative stress gradient;
S3, defining a field diameter of a corresponding notch according to different stagnation point positions;
S4, according to a divided field diameter, bringing the field diameter divided into simplified one-dimensional field intensity approach models, and calculating a corresponding equivalent stress according to the divided field diameter; and
S5, combining an S-N curve of a smooth material, and predicting the fatigue life of the notched component according to a calculated equivalent stress.

Further, a stress selection on the most dangerous path refers to various influencing factors, including notch shape and geometric size, and the relative stress gradient refers to a supporting effect brought by notch morphology.

Further, a formula for calculating the relative stress gradient is:

$${χ}^{*} ( r ) = \frac{1}{σ ( r , θ = 0 )} \frac{\partial {σ}_{yy} ( r , θ = 0 )}{\partial r} ;$$

in the above formula, X* represents the relative stress gradient, σ_{yy} represents a stress distribution on a selected path and r represents a distance from the notch root.

Further, the notch types specifically include:
passivation notch: no stagnation point in the relative stress gradient;
sharp notch: a stagnation point exists after a given nominal stress; and
moderate notch: a stagnation point exists before the given nominal stress.

Further, the defining the field diameter of the corresponding notch includes: defining a field diameter of the sharp notch, defining a field diameter of the moderate notch and defining a field diameter of the passivation notch, where
the field diameter of the sharp notch is defined as distance from the notch root to the stagnation point;
the field diameter of the moderate notch is defined as distance from the notch root to the stagnation point; and
the field diameter of the passivation notch is defined as distance from the notch root to the stable stress change.

Further, simplified models of the field intensity approach are expressed as:

$$\begin{matrix}{σ}_{FI} = {σ}_{max} - ξ ( Ω ) , \\ {σ}_{FI} = {σ}_{max} + ξ ( Ω ) , \\ ξ ( Ω ) = \frac{{\int }_{0}^{R} ( {σ}_{max} - {σ}_{r} ) ( \vert \frac{d {σ}_{r} 1}{dr {σ}_{max}} \vert cos θ \vert ) dr}{{\int }_{0}^{R} ( \vert \frac{d {σ}_{r} 1}{dr {σ}_{max}} \vert r ( θ ) cos θ ) dr} ;\end{matrix}$$

in the above formulas, σ_{F1} represents an equivalent stress; σₘₐₓ represents a peak stress at the notch root; ξ(Ω) represents a correction parameter of a main body acting on the target field strength, and is related to a stress distribution in a fatigue failure zone; σᵣ represents a stress distribution on an one-dimensional path, R represents a field diameter defining the corresponding notch, and θ takes 0 for a symmetrical structure.

Further, the passivation notch and the moderate notch adopt σ_{F1}=σₘₐₓ-$(52) to correct the peak stress, and the sharp notch adopts O_{F1}=σₘₐₓ+$ (2) to correct the peak stress.

Further, the predicting the fatigue life of the notched component specifically includes:
obtaining an S-N curve of a corresponding smooth material;
using the calculated equivalent stress as an input parameter of the S-N curve;
finding a point corresponding to the equivalent stress on the S-N curve and reading a corresponding fatigue life; and
determining an expected fatigue life of the notched component under actual working conditions.

Further, following steps are also included in the S1: firstly, carrying out finite element analysis on the notched component according to external load environment and shape parameters of the loaded component, and extracting stress distribution on the path of the notched root.

The disclosure has following beneficial effects.

According to the disclosure, three notch types are divided according to the position of the stagnation point in the relative stress gradient, and the field diameters of the three notch types are defined by the stagnation point, so that any notched component is capable of being divided, and the modification of peak stress is capable of being distinguished according to the notch types, so that more accurate life prediction is capable of being obtained.

On the premise of mastering the S-N curve of the material, the predicted service life is capable of being obtained only by carrying out finite element analysis including elasticity and plasticity without testing the notched component, the operation is simple, the calculation is convenient and the applicability is wide.

### BRIEF DESCRIPTION OF THE DRAWINGS

In order to explain the technical scheme of the present disclosure or the prior art more clearly, the figures needed to be used in the description of the embodiment or the prior art are briefly introduced below. Obviously, the figures in the following description are only a part of the present disclosure, and other figures are capable of being obtained according to these figures without creative work for ordinary technicians in this field.

FIG. 1 is a schematic flow chart of a fatigue life prediction method of a modified field intensity approach based on notch type division according to an embodiment of the present disclosure.

FIG. 2 is a schematic diagram of a passivation notch applied with a relative nominal stress of 270 MPa as defined in an embodiment of the present disclosure.

FIG. 3 is a schematic diagram of a moderate notch applied with a relative nominal stress of 270 MPa as defined in an embodiment of the present disclosure.

FIG. 4 is a schematic diagram of a sharp notch applied with a relative nominal stress of 270 MPa as defined in an embodiment of the present disclosure.

FIG. 5 is a schematic diagram showing fatigue life prediction results of a specific example according to an embodiment of the present disclosure.

### DETAILED DESCRIPTION OF THE EMBODIMENTS

In order to make the purpose, technical scheme and advantages of the disclosure more clearly understood, the disclosure is further explained in detail in combination with specific embodiments.

It should be noted that, unless otherwise defined, technical terms or scientific terms used in the present disclosure should have their ordinary meanings as understood by people with ordinary skills in the field to which the present disclosure belongs. The terms “first”, “second” and the like used in the present disclosure do not indicate any order, quantity or importance, but are only used to distinguish different components. Similar words such as “including” or “comprising” mean that the elements or objects appearing before the word cover the elements or objects listed after the word and their equivalents, without excluding other elements or objects. Similar words such as “connected” or “linked” are not limited to physical or mechanical connection, but can include electrical connection, whether direct or indirect. “Up”, “down”, “left” and “right” are only used to indicate the relative positional relationship. When the absolute position of the described object changes, the relative positional relationship may also change accordingly.

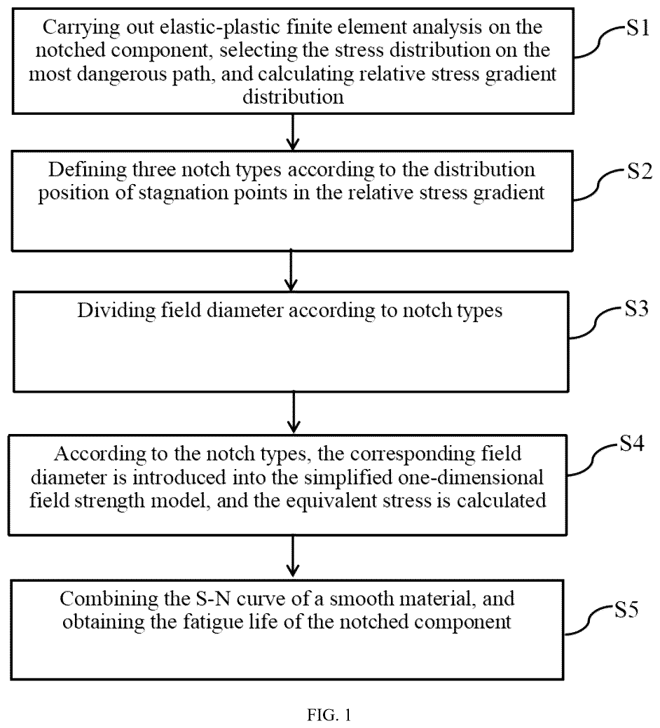

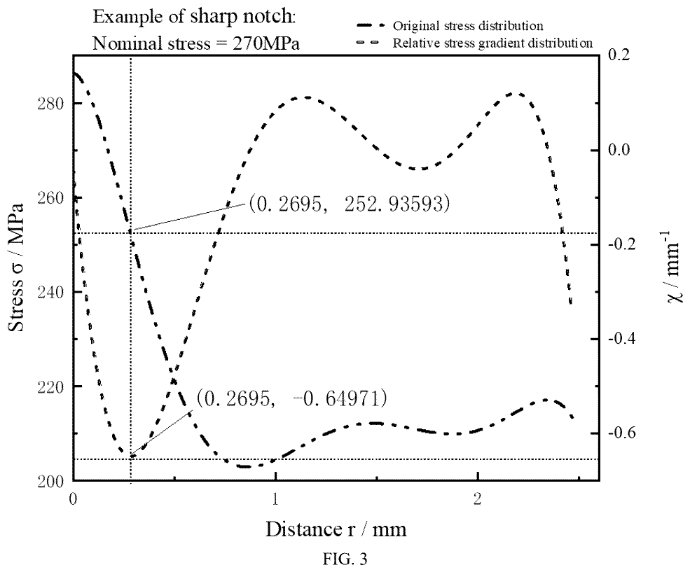

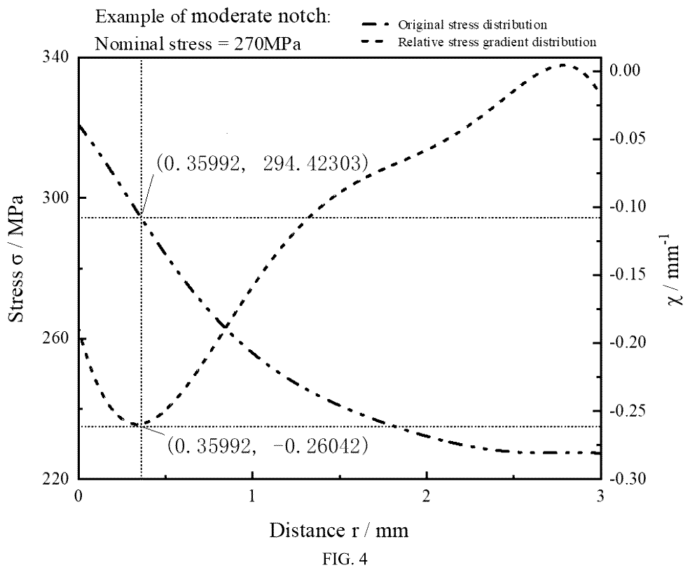

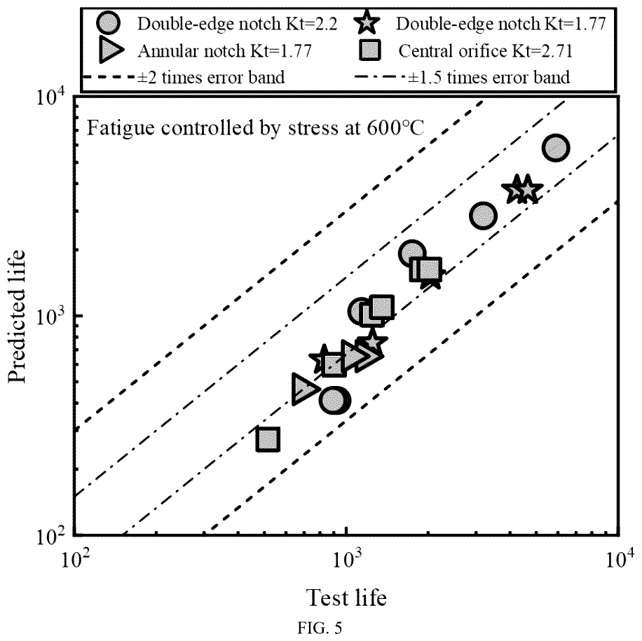

As shown in FIGS. 1-5, a fatigue life prediction method of a modified field intensity approach based on notch type division includes following steps:

S1, firstly, carrying out finite element analysis on a notched component according to external load environment and shape parameters of the loaded component, and then carrying out elastic-plastic finite element analysis on the analyzed notched component to obtain stress-strain distribution, extracting stress distribution on the most dangerous path of the analyzed notched component and calculating a corresponding relative stress gradient; the stress selection of the most dangerous path takes into account influences of notch shape, geometric size and other factors, and the relative stress gradient takes into account an supporting effect brought by notch morphology. A formula for calculating the relative stress gradient is:

$${χ}^{*} ( r ) = \frac{1}{σ ( r , θ = 0 )} \frac{\partial {σ}_{yy} ( r , θ = 0 )}{\partial r} ; \qquad ( 1 )$$

in the above formula, X* represents the relative stress gradient, σ_{yy} represents a stress distribution on a selected path and r represents a distance from the notch root.

The steps proceeds to S2.

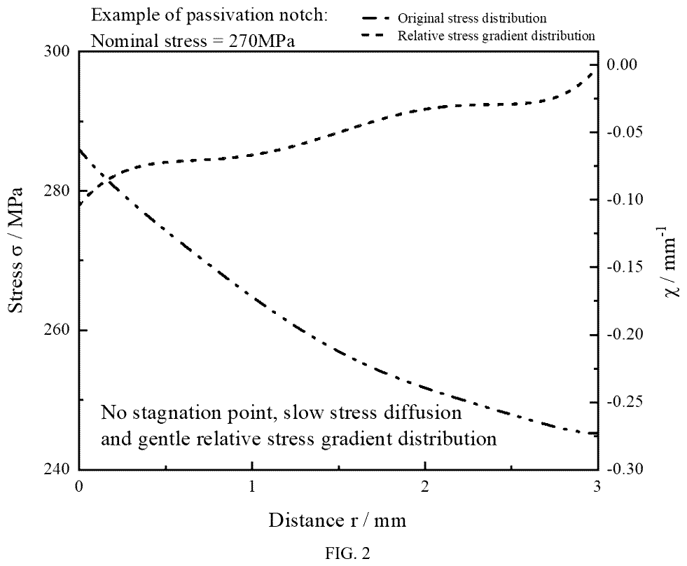

S2: dividing three different notch types according to the obtained relative stress gradient, as defined in FIGS. 2-4. Passivation notch: no stagnation point in the relative stress gradient; sharp notch: a stagnation point exists after a given nominal stress; and moderate notch: a stagnation point exists before the given nominal stress; divided according to the stagnation point position of the relative stress gradient, the three different notch types represent: the stress gradient distribution before the stagnation point is more radical, but the stress gradient changes after the stagnation point; for the passivation notch: there is no stagnation point in the relative stress gradient, representing that the change of the whole stress gradient is slow; for the sharp notch: the stagnation point exists after the given nominal stress, representing that the corresponding stress gradient before the stagnation point is in a more radical change; finally, for the moderate notch, the stagnation point exists before the given nominal stress, representing that only the stress change before this distance needs to be considered.

The steps proceeds to S3.

S3, according to different stagnation point positions, defining field diameters of the sharp notch and the moderate notch as distance from the notch root to the stagnation point, and defining the field diameter of passivation notch as distance from the notch root to the stable stress change. Details are shown in FIGS. 2-4.

The steps proceeds to S4.

S4, bringing the field diameter divided in the S3 into simplified one-dimensional field intensity approach models, and defining the field diameter R as described in the S2. The simplified models of the field intensity approach are as follows:

$${σ}_{FI} = {σ}_{max} - ξ ( Ω ) , \qquad ( 2 )$$

$${σ}_{FI} = {σ}_{max} + ξ ( Ω ) , \qquad ( 3 )$$

$$ξ ( Ω ) = \frac{{\int }_{0}^{R} ( {σ}_{max} - {σ}_{r} ) ( \vert \frac{d {σ}_{r} 1}{dr {σ}_{max}} \vert cos θ \vert ) dr}{{\int }_{0}^{R} ( \vert \frac{d {σ}_{r} 1}{dr {σ}_{max}} \vert r ( θ ) cos θ ) dr} ; \qquad ( 4 )$$

in the above formulas, OF represents an equivalent stress; σₘₐₓ represents a peak stress at the notch root; ξ (Ω) represents a correction parameter of a main body acting on the target field strength, and is related to a stress distribution in a fatigue failure zone; σᵣ represents a stress distribution on an one-dimensional path, R represents a field diameter defining the corresponding notch, and θ takes 0 for a symmetrical structure. For the passivation notch and the moderate notch, the modification of peak stress satisfies Formula (2), and for the sharp notch, the modification of peak stress satisfies Formula (3).

The steps proceeds to S5.

S5, combining an S-N curve of a smooth material, and a life corresponding to the calculated equivalent stress is the fatigue life of the notched component.

Concrete steps for predicting the fatigue life of the notched component are as follows:
obtaining an S-N curve of a corresponding smooth material;
using the calculated equivalent stress as an input parameter of the S-N curve;
finding a point corresponding to the equivalent stress on the S-N curve and reading a corresponding fatigue life; and
determining an expected fatigue life of the notched component under actual working conditions.

Specific examples are as follows:

The fatigue life prediction method of a modified field intensity approach based on notch type division of the disclosure is used to predict the cycle life of any notched component with different conditions and different materials under stress-controlled fatigue load.

The stress control fatigue test of 21 groups of 316H stainless steel at 600° C. is carried out, and the 21 groups of data include four notch samples with different notch types and three different stress concentration factors. The applied stress is nominal stress, and the stress ratio is −1. The test methods all comply with the national standard GBT3075-2020 “Metallic materials-Fatigue testing-Axial-force-controlled method”.

A low-cycle fatigue test of six groups of 316H stainless steel specimens with central orifice is carried out. The stress concentration coefficient of the specimens is 2.71, and the stress amplitudes are 220 MPa, 230 MPa, 240 MPa, 270 MPa and 300 MPa. A low-cycle fatigue test of six groups of 316H stainless steel specimens with double-edge notch is carried out. The stress concentration coefficient of the specimens is 2.2, and the stress amplitudes are 230 MPa, 240 MPa, 270 MPa, 300 MPa and 310 MPa. A low-cycle fatigue test of six groups of 316H stainless steel specimens with double-edge notch is carried out. The stress concentration coefficient of the specimens is 1.77, and the stress amplitudes are 240 MPa, 270 MPa, 300 MPa, 320 MPa and 340 MPa. The low cycle fatigue test of three groups of 316H stainless steel ring samples shows that the stress concentration coefficient of the specimens is 1.77, and the stress amplitudes are 240 MPa and 270 MPa. The test method is as described in the specification.

Firstly, according to the S1 of the disclosure, according to the elastic-plastic finite element analysis, the tensile stress-strain distribution under nominal stress is obtained, the most dangerous path is selected, its stress distribution is extracted, and the relative stress gradient distribution on this path is calculated; then, according to the S2, three notch types are divided; then, according to the S3, the field diameters of different notched components are determined; according to the S4, the equivalent stress of the simplified one-dimensional field intensity approach model is calculated; finally, according to the S5, combined with the S-N curve of the smooth material, the life corresponding to the calculated equivalent stress is the fatigue life of the notched component.

For different notch types and various stress concentration factors, the life predicted by the disclosure is within 2 times of the error band. Therefore, the life prediction model of the disclosure has universality for different test materials, different test loads, different notch types and different stress concentration factors.

It should be understood by those skilled in the art that the discussion of any of the above embodiments is only exemplary, and it is not intended to imply that the scope of the present disclosure is limited to these examples; Under the idea of the present disclosure, the technical features in the above embodiments or different embodiments can also be combined, and the steps can be realized in any order, and there are many other variations in different aspects of the present disclosure as described above, which are not provided in the details for brevity.

The present disclosure is intended to cover all such alternatives, modifications and variations that fall within the broad scope of the claims. Therefore, any omission, modification, equivalent substitution, improvement, etc. within the spirit and principle of the disclosure should be included in the protection scope of the disclosure.

What is claimed is:

1. A fatigue life prediction method of a modified field intensity approach based on notch type division, comprising following steps:
S1, carrying out elastic-plastic finite element analysis on a notched component to be analyzed, obtaining a stress-strain distribution, extracting a stress distribution on the most dangerous path of the notched component, and calculating a corresponding relative stress gradient;
S2, dividing notch types according to an obtained relative stress gradient;
S3, defining a field diameter of a corresponding notch according to different stagnation point positions;
S4, according to a divided field diameter, bringing the field diameter divided into simplified one-dimensional field intensity approach models, and calculating a corresponding equivalent stress according to the divided field diameter; and
S5, combining an S-N curve of a smooth material, and predicting the fatigue life of the notched component according to a calculated equivalent stress.

2. The fatigue life prediction method of the modified field intensity approach based on notch type division according to claim 1, wherein a stress selection on the most dangerous path refers to various influencing factors, comprising notch shape and geometric size, and the relative stress gradient refers to a supporting effect brought by notch morphology.

3. The fatigue life prediction method of the modified field intensity approach based on notch type division according to claim 2, wherein a formula for calculating the relative stress gradient is:
$${χ}^{*} ( r ) = \frac{1}{σ ( r , θ = 0 )} \frac{\partial {σ}_{yy} ( r , θ = 0 )}{\partial r} ;$$
in the above formula, x* represents the relative stress gradient, Oyy represents a stress distribution on a selected path and r represents a distance from the notch root.

4. The fatigue life prediction method of the modified field intensity approach based on notch type division according to claim 3, wherein the notch types specifically comprise:
passivation notch: no stagnation point in the relative stress gradient;
sharp notch: a stagnation point exists after a given nominal stress; and
moderate notch: a stagnation point exists before the given nominal stress.

5. The fatigue life prediction method of the modified field intensity approach based on notch type division according to claim 4, wherein the defining the field diameter of the corresponding notch comprises: defining a field diameter of the sharp notch, defining a field diameter of the moderate notch and defining a field diameter of the passivation notch, wherein
the field diameter of the sharp notch is defined as distance from the notch root to the stagnation point;
the field diameter of the moderate notch is defined as distance from the notch root to the stagnation point; and
the field diameter of the passivation notch is defined as distance from the notch root to the stable stress change.

6. The fatigue life prediction method of the modified field intensity approach based on notch type division according to claim 5, wherein simplified models of the field intensity approach are expressed as:
$$\begin{matrix}{σ}_{FI} = {σ}_{max} - ξ ( Ω ) , \\ {σ}_{FI} = {σ}_{max} + ξ ( Ω ) , \\ ξ ( Ω ) = \frac{{\int }_{0}^{R} ( {σ}_{max} - {σ}_{r} ) ( \vert \frac{d {σ}_{r} 1}{dr {σ}_{max}} \vert cos θ \vert ) dr}{{\int }_{0}^{R} ( \vert \frac{d {σ}_{r} 1}{dr {σ}_{max}} \vert r ( θ ) cos θ ) dr} ;\end{matrix}$$
in the above formulas, σ_{F1} represents an equivalent stress; Omax represents a peak stress at the notch root; ξ(Ω) represents a correction parameter of a main body acting on the target field strength, and is related to a stress distribution in a fatigue failure zone; σᵣ represents a stress distribution on an one-dimensional path, R represents a field diameter defining the corresponding notch, and θ takes 0 for a symmetrical structure.

7. The fatigue life prediction method of the modified field intensity approach based on notch type division according to claim 6, wherein the passivation notch and the moderate notch adopt σ_{F1}=σₘₐₓ−ξ(Ω) to correct the peak stress, and the sharp notch adopts σ_{F1}=σₘₐₓ+ξ(Ω) to correct the peak stress.

8. The fatigue life prediction method of the modified field intensity approach based on notch type division according to claim 7, wherein the predicting the fatigue life of the notched component specifically comprises:
obtaining an S-N curve of a corresponding smooth material;
using the calculated equivalent stress as an input parameter of the S-N curve;
finding a point corresponding to the equivalent stress on the S-N curve and reading a corresponding fatigue life; and
determining an expected fatigue life of the notched component under actual working conditions.

9. The fatigue life prediction method of the modified field intensity approach based on notch type division according to claim 1, wherein following steps are also comprised in the S1: firstly, carrying out finite element analysis on the notched component according to external load environment and shape parameters of the loaded component, and extracting stress distribution on the path of the notched root.
